(19) United States
(12) Patent Application Publication
(10) Pub. No.: US 2024/0170105 A1
(43) Pub. Date: May 23, 2024

(54) ESTIMATION OF CHEMICAL PROCESS OUTPUTS BY SINGLE OBJECT FEEDSTOCK HYPERSPECTRAL IMAGING

(71) Applicant: X Development LLC, Mountain View, CA (US)

(72) Inventors: Aleksandra Spyra, Mountain View, CA (US); Diosdado Banatao, Mountain View, CA (US); Clare Lee, Palto Alto, CA (US); Allen Zhao, Mountain View, CA (US); Gearoid Murphy, San Jose, CA (US); Daniel Rosenfeld, Mountain View, CA (US); Alexander Holiday, Brookfield, WI (US)

(73) Assignee: X Development LLC, Mountain View, CA (US)

(21) Appl. No.: 18/516,735

(22) Filed: Nov. 21, 2023

Related U.S. Application Data
(60) Provisional application No. 63/427,535, filed on Nov. 23, 2022.

(51) Int. Cl. G16C 20/30; G16C 20/70

(52) U.S. Cl. CPC G16C 20/30; G16C 20/70

(57) ABSTRACT

An intermediate data set can be generated based on an image of a set of objects, where each of the set of objects includes a plastic. A predicted chemometric property can be generated by inputting the intermediate data set to a machine-learning model. The chemometric property can be of a physical output pyrolysis oil produced by performing a pyrolysis processing of the set of objects using a pyrolysis reactor. A result associated with the set of objects can be generated, where the result is based on or includes the predicted chemometric property.

### CROSS REFERENCE TO RELATED APPLICATIONS

This application claims the benefits of priority to U.S. Provisional Application No. 63/427,535, filed on Nov. 23, 2022, entitled “Estimation Of Chemical Process Outputs By Single Object Feedstock Hyperspectral Imaging,” the entirety of which is incorporated herein by reference.

### TECHNICAL FIELD

This specification generally relates to facilitating high-quality downstream processing (e.g., pyrolysis processing) of sets of objects (e.g., automatically sorted objects) by analyzing one or more hyperspectral images using one or more machine-learning models to predict chemical process outputs (e.g., chemokinetic properties of a pyrolysis oil produced from the set of objects). The specification further relates to identifying particular features of images of one or more objects that are predictive of one or more quality metrics of a downstream chemical process.

### BACKGROUND

As much as five billion metric tons of plastic waste currently sit in landfills worldwide, and by 2040 another 7.7 billion metric tons of plastic will be mismanaged (landfilled, incinerated, or leaked) and not recycled. These waste streams represent $5 trillion in commodity value if there was a way to economically re-enter the materials into the economy, with the added benefit of reducing gigaton-scale levels of CO₂ emissions and creating an impactful and economically viable circular economy.

During recycling workflows, sets of objects can be fed to one or more mechanical or chemical processing lines to produce physical outputs. Mechanical recycling is the dominant strategy for recycling plastic and involves grinding, melting, and re-extruding plastic waste. Recycling facilities are frequently designed to process streams of sorted materials with high purity, to retain a high level of material performance in recycled products. However, feedstock impurity reduces the effectiveness of recycling, due to complex formulations with additives, as well as the physical degradation of materials, even just after a few cycles of mechanical recycling. For example, with plastic materials, polylactic acid (PLA) is a common waste plastic often undetected in polyethylene terephthalate (PET) sorting and mechanical recycling operations. As another example, chlorinated compounds such as polyvinyl chloride (PVC) are not tolerated in both mechanical and chemical recycling operations, because corrosive compounds are produced during recycling processes, which limits the value of hydrocarbon outputs.

Mechanical recycling is limited in its applicability to mixed, composite, and contaminated waste streams, in part because it employs mechanical separation and reformation processes that are (sensitive to chemical contaminants and may be unable to modify the chemical structure of waste material.

Chemical recycling may resolve some limitations of mechanical recycling by breaking the chemical bonds of waste materials into smaller molecules. For example, in the case of polymeric materials, chemical recycling may provide an avenue to recover oligomers, or even basic molecules (e.g., monomers) from a plastic waste feedstock. In the case of polymers, chemical recycling processes may include operations to depolymerize and dissociate the chemical makeup of a complex plastic product, such that its by-products can be up-cycled into feedstocks for new materials. Elements of chemical recycling may permit a material to be repeatedly dissociated into primary feedstock materials. In this way, rather than being limited by chemical structure and material integrity to a limited number of plastic types, as in mechanical recycling, chemical recycling may be integrated into an ‘end-to-end’ platform to facilitate reuse of molecular components of recyclable materials. For example, the products of chemical recycling may include basic monomers (ethylene, acrylic acid, lactic acid, vinyl, etc.), feedstock gases (carbon monoxide, methane, ethane, etc.), or elemental materials (sulfur, carbon, etc.). Instead of being limited to a single group of recycled products, based on the molecular structure of the input waste material, products may be identified that can be synthesized from intermediary chemicals that can be produced from the waste by chemical reactions. In so doing, the end-to-end platform may manage a waste stream by generating a chemical reaction scheme to convert the waste material into one or more target products. For example, the end-to-end platform may direct different portions of waste feedstock to different chemical recycling facilities, for chemical conversion into a variety of target products.

However, the efficiency of recycling (e.g., chemical recycling) and the reliability of outputs of the recycling still depends on the degree to which the objects received for processing correspond to one or more target materials, have limited contamination, etc. Further, the degree to which an output of the recycling has target characteristics depends on the degree to which a workflow can ensure that feedstock being recycled meets certain quality constraints.

Meanwhile, individual objects initially received in a recycling line are frequently not composed of purely one material. For example, a plastic container may include multiple types of plastic, a label (that includes paper), and food contaminants. Routing such objects to a downstream process (e.g., a downstream pyrolysis process) may result in sub-optimal results. For example, a yield of a target liquid produced by processing contaminated objects via pyrolysis may be relatively low, an amount of char produced may be relatively high, etc.

Thus, it would be advantageous to implement improved techniques that result in improved outputs from downstream processing lines, even when objects in initial feedstock are diverse, potentially composed of multiple materials, and potentially contaminated (e.g., with food, dirt, etc.).

### SUMMARY

In some instances, a computer-implemented method is provided that includes: generating an intermediate data set based on an image of or line scans of a set of objects, wherein each of the set of objects includes a plastic; generating a predicted chemometric property of a physical output pyrolysis oil produced by processing the set of objects using a pyrolysis reactor, wherein the predicted chemometric property is generated by inputting the intermediate data set to a machine-learning model; and generating a result associated with the set of objects, wherein the result is based on or includes the chemometric property.

Generating the intermediate data set may include generating a hypercube based on a set of line scans of the line scans of the set of objects, a first dimension of the hypercube may correspond to a first spatial dimension in a real-world space, a second dimension of the hypercube may correspond to a second spatial dimension in the real-world space, and a third dimension of the hypercube may correspond to a frequency dimension, and values in the hypercube represent at least one of an intensity, a power, a reflectance, a transmittance, an absorbance, and a trans-reflectance.

Generating the intermediate data set may include generating, for each material of a set of materials, a predicted relative or absolute amount of the material in the set of objects.

Generating the intermediate data set may include generating, for each material of a set of materials, a portion of a weight or mass of the set of the objects that is predicted to be attributed to the material.

The predicted chemometric property of the pyrolysis oil may be an American Petroleum Institute (API) gravity, density, or relative density of the pyrolysis oil.

The predicted chemometric property of the pyrolysis oil may be a vapor pressure of a crude oil produced using the pyrolysis oil.

The predicted chemometric property of the pyrolysis oil may be a pour point of the pyrolysis oil.

The predicted chemometric property of the pyrolysis oil may be or may be based on an amount of one or more halogens in the pyrolysis oil.

The predicted chemometric property of the pyrolysis oil may be or may be based on an amount of inorganic contaminants and organic contaminants in the pyrolysis oil. The inorganic contaminants may comprise at least one of sulfur, chlorine, and phosphorus. The organic contaminants may comprise at least one of sulfur, Polyfluorinated Substances (PFAS), caprolactams, organic acids, perflourinated and flourinated compounds, halogenated organic compounds, and oxygen measured by neutron activation.

The method may further include: controlling whether the set of objects are routed towards a pyrolysis-process pipeline based on the result.

The result may include a selection or identification of one or more other objects to combine with the set of objects before the pyrolysis processing is performed on the set of objects.

The image of the set of objects may be generated based on a set of line scans obtained at different wavelengths.

The wavelengths may be selected from a group of wavelength ranges consisting of 1000-1700 nm, 2200-5000 nm, and 400-1000 nm.

The image of the set of objects may be generated by performing one of a line scan, an area scan, and a point mapping.

Generating the intermediate dataset may include performing, for each material of a set of materials, hydrocarbon analysis on the set of the objects. The hydrocarbon analysis may provide a profile of at least one paraffins, iso-paraffins, and aromatics present in the set of the objects.

Generating the intermediate data set may include performing, for each material of a set of materials, simulated distillation of the set of the objects. The simulated distillation may provide volumetric distillation profiles of the set of the objects.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

The details of one or more implementations of the subject matter described in this specification are set forth in the accompanying drawings and the description below. Other potential features, aspects, and advantages of the subject matter will become apparent from the description, the drawings, and the claims.

### BRIEF DESCRIPTION OF THE DRAWINGS

The present disclosure is described in conjunction with the appended figures:

FIG. 1 is a block diagram of an example system 100 implemented to perform estimation of chemical process outputs.

FIG. 2 illustrates a process stream where a machine-learning model is trained to process image data to predict characteristics of feedstocks.

FIG. 3 identifies exemplary specifications for 30 batches to use to generate training data to train a model.

FIG. 4 identifies exemplary specifications for 16 batches to use to generate training data to train a model.

FIG. 5 illustrates an example where three hyperspectral images of objects were used to predict pyrolysis outputs of a batch that includes the images.

### DETAILED DESCRIPTION

According to one innovative aspect of the subject matter described in this specification, image data of a feedstock can be collected. The image data may include an image or one or more line scans. In one implementation, the image may be generated based on a set of line scans of a set of objects obtained at different wavelengths. The wavelengths may be selected from a group of wavelength ranges consisting of 1000-1700 nm, 2200-5000 nm, and 400-1000 nm. In another implementation, the image of the set of objects may be generated by performing one of a line scan, an area scan, and a point mapping. In some instances, the image data includes hyperspectral data and/or a hyperspectral cube (e.g., that includes a first spatial dimension, a second spatial dimension, and a frequency dimension and that indicates—for each of a set of points in the three-dimensional space—an intensity, a power, a reflectance, a transmittance, an absorbance, and a trans-reflectance).

At least part of the image data can then be fed to a machine-learning model that can generate a predicted chemometric property of a physical output of a downstream processing line, where the physical output is one predicted to be produced if the object(s) represented in the at least part of the image data are transformed using the downstream processing line. For example, the machine-learning model may generate a predicted chemometric property of a pyrolysis oil produced by performing a pyrolysis process using the object(s). Exemplary chemometric properties of a pyrolysis oil include: an American Petroleum Institute (API) gravity of, a density of, a relative density of, a pour point of, an amount of a halogen in the pyrolysis oil, an amount of inorganic contaminant (e.g., sulfur, chlorine, phosphorus, etc.) in the pyrolysis oil, an amount of organic contaminants (e.g. sulfur, Polyfluorinated Substances (PFAS), caprolactams, organic acids, perflourinated and flourinated compounds, halogenated organic compounds, and oxygen measured by neutron activation), etc. An overall quality metric or classifier is derived by an aggregate of an individual chemometric property or other predictive functions. Another exemplary chemometric property of a pyrolysis oil is a vapor pressure of a crude oil produced using the pyrolysis oil.

The at least part of the image data fed to the machine-learning model may represent a single object or multiple objects. For example, in some instances, the image data is fed to a segmentation model that predicts which portions of the image data correspond to distinct types of material (e.g. PET bottle, paper label, PP bottle cap). The at least part of the image data may then be defined to include one or more portions of the image data that correspond to (for example) a single object or a particular set of objects.

In some instances, other processing is performed to identify the single object or the particular set of objects to be represented in the at least part of the image data. For example, the other processing may include predicting a compositional attribute of each of one or more objects (e.g., an absolute or relative amount of an object that is of a given material and/or determining that the absolute or relative amount exceeds a predefined threshold). The other processing may include predicting a weight or mass of a specific type of material (e.g., a specific type of plastic) or of a specific material (e.g., a specific plastic) in the object and/or predicting a total weight or mass of the object. Thus, an absolute or relative weight or mass of the specific type of material may be predicted.

In some instances, other processing is performed to determine a profile of at least one of paraffins, iso-paraffins, and aromatics present in the set of the objects. The profile may be determined by performing hydrocarbon analysis (DHA) on the set of objects. The purpose of detailed DHA is to determine the bulk hydrocarbon group type composition, such as PONA: Paraffins, Olefins, Naphthenes, and Aromatics.

In some instances, other processing is performed to determine volumetric distillation profiles of the set of objects. The volumetric distillation profiles may be determined by performing simulated distillation of the set of the objects. Simulated distillation may be a gas chromatographic method and may be evolved into an indispensable tool in petroleum industries to determine the distillation behavior of different petroleum products and to ensure fuel quality. In contrast to classic physical distillation, simulated distillation may exhibit a range of advantages that include comparatively very small sample amounts and the possibility of automation, which are of great importance in fields of research and in the development of novel fuels.

The other processing may include predicting or determining whether to include the object in a particular processing stream (e.g., based on the predicted weight or mass of the specific type of material), whether to complete the particular processing stream (that ingests at least the object), how to tune a parameter to decide which other object(s) to include for ingest in the particular processing stream, how to define a parameter for the processing stream, etc.

This determination may be based on which other objects are currently assigned or tentatively assigned to a same lot or same feedstock for a processing line that is associated with the specific material (e.g., to extract the specific material). The determination may include comparing the predicted absolute or relative weight or mass of the specific type of material to a threshold. The threshold may be specific to a given processing line and may be determined based on (for example) input from a client system, one or more recent outputs from the processing line, and/or composition data for one or more other objects already assigned to or routed to the processing line. For example, a threshold may be determined by identifying multiple other objects flagged to proceed into a next feedstock or next lot for a processing stream and determining a threshold for the object based on a predefined criteria for the processing stream, an estimated total weight or total mass of the one or more other objects and/or an estimated weight or mass of the specific type of material or of the specific material in the one or more other objects. In some instances, the threshold further depends on an estimated total weight or mass of the object.

To illustrate, at a given point in time and for a given processing line, 10 objects may have already been routed, may have been assigned to, or may have been tentatively assigned to a given processing line. Those 10 objects may have a cumulative predicted or actual mass of m₁ and a cumulative predicted target mass of t₁. The processing line may be configured to receive a lot of approximately mass m₂ and may require a target mass of at least a target-mass threshold t₂ (with the target mass corresponding to a target absolute or weighted mass of one or more particular materials or material types and/or with a total mass corresponding to a total target absolute or total weighted mass of one or one or more particular materials or material types). A given object may have a predicted mass of m₂-m₁ (e.g., corresponding to a total mass of the object, a total mass of materials of a given type in the object, or a mass of a particular material in the object), and there may be a predicted target mass of t₃. It may be determined that the given object is to be added to the lot if the sum of the cumulative predicted target mass t₁ and the predicted mass of the object meets or exceeds the target-mass threshold t₂. Otherwise, the given object may be routed to a different processing line or to a discard bin. In some instances, for each object, a ratio of the predicted target mass to the predicted total mass is evaluated and compared to a threshold. However, the threshold may change as various objects are added to a lot. To illustrate, if the ratio is very high for a first group of objects added to the lot, a lower ratio may be tolerable for a second group of objects added to the lot, since the lot in its entirety could still meet a target overall ratio.

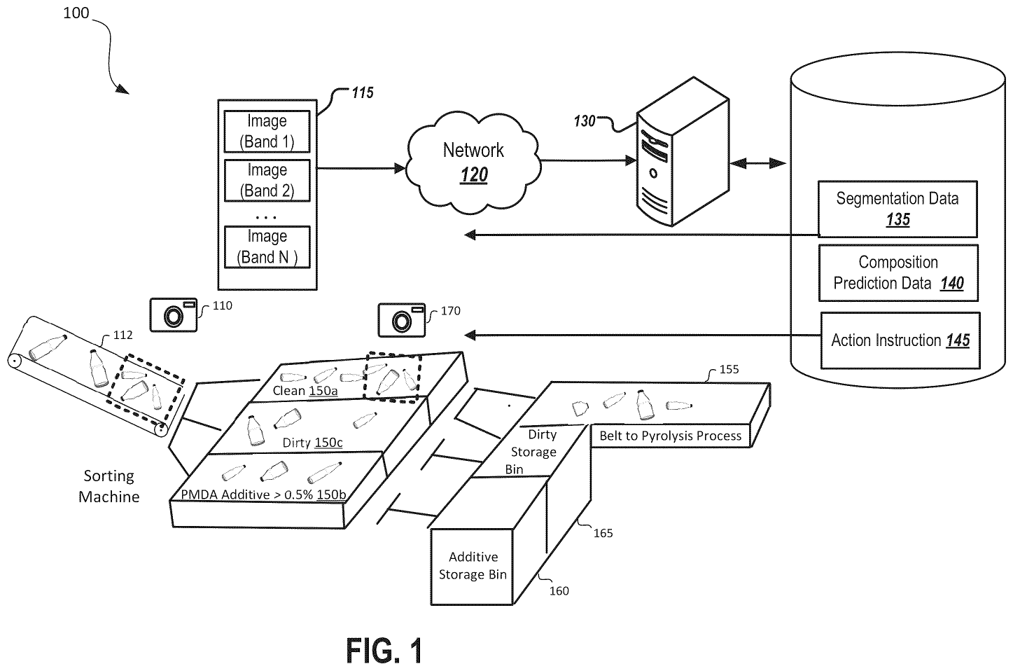

FIG. 1 is a block diagram of an example system 100 implemented to perform estimation of chemical process outputs. The system 100 includes a camera system 110 for capturing images (e.g., hyperspectral images) or line scans (e.g., hyperspectral line scans) of objects. Each image may be of part or all of one or more objects that are being moved by a conveyor belt 112. The objects may include those that were initially collected from multiple individuals' recycling bins.

It will be appreciated that an image or a line scan collected by the camera system 110 and analyzed may include a non-hyperspectral image, and disclosures herein that refer to a hyperspectral image may be adapted to use non-hyperspectral image. For example, the camera system 110 may include a lens with low absorption in the visible range, near infrared range, short-wave infrared range, or mid-wave infrared range. Thus, the captured image may depict signals in the visible range, near infrared range, short-wave infrared range, or mid-wave infrared range, respectively. The camera system 110 can include any of a variety of illumination sources, such as a light emitting diode (which may be synchronized to the camera sensor exposure), incandescent light source, laser, and/or black-body illumination source. A light emitting diode included in camera system 110 may have a peak emission the coincides with a peak resonance of a given chemical of interest (e.g., a given type of plastic) and/or a bandwidth that coincides with multiple molecular absorptions. In some instances, multiple LEDs are included in camera system 110, such that a given specific spectral region can be covered. LED light sources provide coherent light, and controlling emission and signal to noise may be more feasible than for other light sources. Further, they are generally more reliable and consume less power relative to other light sources.

In some instances, camera system 110 is configured such that an optical axis of a lens or image sensor of the camera is between 75-105 degrees, 80-90 degrees, 85-95 degrees, 87.5-92.5 degrees, 30-60 degrees, 35-55 degrees, 40-50 degrees, 42.5-47.5 degrees, or less than 15 degrees relative to a surface supporting the object(s) being imaged (e.g., a conveyor belt). In some instances, the optical system includes multiple cameras, where an angle between an optical axis of a first camera relative to a surface supporting the object(s) is different than an angle between an optical axis of a second camera relative to the surface. The difference may be (for example) at least 5 degrees, at least 10 degrees, at least 15 degrees, at least 20 degrees, at least 30 degrees, less than 30 degrees, less than 20 degrees, less than 15 degrees, and/or less than 10 degrees. The difference may facilitate detecting signals from objects having different shapes or being positioned at different angles relative to an underlying surface (e.g., having different tilts). In some instances, a first camera filters for a different type of light relative to a second camera. For example, a first camera may be an infrared camera, and a second camera may be a visible-light camera. In some instances, camera system 110 includes a light source that is in a specular reflection condition relative to the camera and a second light source in a diffuse reflection condition relative to the camera (e.g., facilitate detecting objects with different specular and diffuse reflectances).

The camera system 110 may include a light guide (e.g., of a fiber-optic, hollow, solid, or liquid-filled type) to transfer light from an illumination source to an imaging location, which can reduce heat released at the imaging location. The camera system 110 may include a type of light source or light optics such that light from the light source(s) is focused to a line or is focused to match a projected size of an entrance slit to a spectrograph. The camera system 110 may be configured such that an illumination source and imaging device (camera) are arranged in a specular reflection condition (so as to generate a bright-field image), in a non-specular (or diffuse) condition (so as to generate a dark-field image) or a mixture of the conditions. Most hyperspectral images have image data for each of several or even dozens of wavelength bands depending on the imaging technique. In many applications, it is desirable to reduce the number of bands in a hyperspectral image to a manageable quantity because processing images with high numbers of bands is computationally expensive (resulting in delay in obtaining results and high power use), because the high dimensional space may prove infeasible to search or have unsuitable distance metrics (“the curse of dimensionality”), or because the bands are highly correlated (“the problem of correlated regressors”). Many different dimensionality reduction techniques have been presented in the past such as principal component analysis (PCA) and pooling. However, these techniques often still carry significant computational cost, require specialized training, and do not always provide the desired accuracy in applications such as image segmentation. In addition, many techniques still attempt to use most or all bands for segmentation decisions, despite the different wavelength bands often having dramatically different information value for segmenting different types of boundaries (e.g., boundaries of different types of regions having different properties, such as material, composition, structure, texture, etc.). This has traditionally led to inefficiency of processing image data for more wavelength bands than are needed for a segmentation analysis. It has also limited accuracy as data for bands that have low relevance to a segmentation boundary obscure key signals in the data with noise and marginally relevant data. In some embodiments, techniques disclosed herein may generate learned embeddings using machine-learning models (e.g., auto-encoders).

Thus, in some instances, a band selection technique may be performed, which may include (for example) one or more actions disclosed in U.S. application Ser. No. 17/811,766, which was filed on Jul. 11, 2022, and which is hereby incorporated by reference in its entirety for all purposes.

To illustrate, in some implementations, synthetic bands or altered bands are generated. The synthetic bands can be generated by processing the image data for one or more of the bands selected in a first iteration. For example, each band within the subset of bands can undergo one or more operations (e.g., image processing operations, mathematical operations, etc.), which can include one or more operations that combine data from two or more different bands (e.g., of those selected in the first iteration). Each of various predetermined functions can be applied to the image data for different combinations of the selected bands (e.g., for each pair of bands or each permutation within the selected subset of bands). This can create a new set of synthetic bands each representing a different modification to or combination of bands selected in the first iteration. The synthetic bands can (for example) additionally or alternatively be derived from convolutions or projections applied to the image data, which are functions that map a group of pixels and single pixels, respectively, to single numbers. A single convolution, multiple convolutions, a single projection and/or multiple projections can be applied across the entire image to create new synthetic bands.

One or more original and/or one or more synthetic bands can be evaluated and/or scored to determine the level to which they are predicted to provide information regarding a target of interest. For example, the band(s) can be evaluated and/or scored based on an extent to which intensities within the bands are predictive of whether and/or an extent to which a corresponding depicted object or a corresponding group of object included a particular type of material. The selected bands may then subsequently be used to analyze other images (e.g., to predict composition characteristics).

In some instances, an image has two dimensions that represent spatial dimensions (e.g., corresponding to a width and length axis) and another dimension that represents different wavelength (or frequency) bands. In some instances, an image is generated based on a set of line scans (e.g., where each line scan may generate an output that identifies an intensity for each position along one spatial dimension and for each of multiple wavelength bands). For example, a line scan can be generated by scanning across a width dimension of the conveyor belt 112 (e.g., for each of the multiple wavelength bands). The conveyor belt may be moving, such that a next line scan is scanning different materials. Multiple line scans can then be combined to generate an image that corresponds to two different dimensions (e.g., and multiple frequency bands).

Thus, for each collected image, image data 115 may be generated that identifies—for each of multiple position and for each of multiple wavelength bands—an intensity, a power, a reflectance, a transmittance, an absorbance, and a trans-reflectance. The image data 115 can be sent over a network 120 to a computing system 130, which can the process the image data 115 (e.g., to potentially perform segmentation, to identify a sorting instruction for individual objects or groups of objects, to predict an output of a processing line if it is used to process a particular group of objects, etc.). In the example of FIG. 1, the camera system 110 includes or is associated with a computer or other device that can communicate over a network 120 with a computing system 130 that processes hyperspectral image data and returns segmented images or other data derived from the segmented images. In other implementations, the functions of the computing system 130 (e.g., to generate profiles, to process hyperspectral image data, to perform segmentation, etc.) can be performed locally at the location of the camera system 110. For example, the system 100 can be implemented as a standalone unit that houses the camera system 110 and the computing system 130.

The network 120 can include a local area network (LAN), a wide area network (WAN), the Internet or a combination thereof. The network 120 can also comprise any type of wired and/or wireless network, satellite networks, cable networks, Wi-Fi networks, mobile communications networks (e.g., 3G, 4G, and so forth) or any combination thereof. The network 120 can utilize communications protocols, including packet-based and/or datagram-based protocols such as internet protocol (IP), transmission control protocol (TCP), user datagram protocol (UDP), or other types of protocols. The network 120 can further include a number of devices that facilitate network communications and/or form a hardware basis for the networks, such as switches, routers, gateways, access points, firewalls, base stations, repeaters or a combination thereof.

The computing system 130 can be configured to use one or more techniques and/or one or more models (e.g., one or more machine-learning models) to process the image data 115. The one or more models may include (for example) a neural network, convolutional neural network, deep neural network, clustering algorithm, etc.

In some instances, the computing system 130 may initially use a segmentation machine-learning model to process image data to predict segmentation data 135. The segmentation data 135 may include a predication as to which data points in the image data depict or represent points or voxels within each of multiple individual objects (or do not represent any objects). For example, if image data corresponds to a static two-dimensional physical space (e.g., and to multiple wavelength bands), the segmentation data may identify various portions within the two-dimensional space in which a given corresponding object is located. To illustrate, the segmentation data 135 may identify—across a two-dimensional grid—a value for each point in the grid, where the value identifies an identifier (e.g., which may be generated using an incremental or pseudorandom technique) of an object predicted as being depicted in the pixel. If it is predicted that no object is depicted in the pixel, a default value (e.g., 0 or a not-a-number value) can be assigned. The segmentation and/or one or more other actions may be performed in accordance with one or more disclosures in U.S. application Ser. No. 17/811,766, which was filed on Jul. 11, 2022, which is hereby incorporated by reference in its entirety for all purposes. In some instances, the segmentation is performed using a trained segmentation machine-learning model and/or one or more segmentation profiles (e.g., which may indicate different hyperspectral data combinations that correspond to different types of materials). The computing system 130 may then store the segmentation data 135 that identifies the portions of the imaged data that are predicted to correspond to various distinct objects.

It will be appreciated that the computing system 130 need not perform a segmentation analysis and/or use a segmentation machine-learning model. For example, hyperspectral data may be collected across an entire extent of one or more axes in the imaging data, which may then be processed (e.g., even if it corresponds to a depiction of multiple objects, portions of objects, multiple at least portions of objections, etc.).

In some instances, the computing system 130 may use a technique (e.g., another technique) or a machine-learning model (e.g., another machine-learning model) to generate—for a given object—composition prediction data 140. The other model may include (for example) a neural network, convolutional neural network, deep neural network, regression model, support vector machine, component analysis (e.g., principal component analysis), etc.

The composition prediction data 140 can associate—for each individually segmented objects or for a collection of full and/or partial objects that were imaged—corresponding predictions as to (for example) an amount of a given material in the object or collection, whether the object or collection includes at least a threshold amount of the given material, whether a condition (e.g., pertaining to a composition having at least a first threshold amount of one material and having less than a second threshold amount of another material) is satisfied, etc.

The composition prediction data 140 may be generated by transforming part or all of the image data 115 using the technique or machine-learning model. For example, the segmentation analysis may predict that a first particular area represents data from a single object. The wavelength data that corresponds to the first particular area can then be transformed using the technique or the machine-learning model to generate a prediction corresponding to an amount of one or more materials in the single object. As another example, segmentation need not be performed, and wavelength data from an entire image may be fed to the technique or machine-learning model.

The computing system can use the composition prediction data 140 to generate one or more action instructions 145. An action instruction may include routing or facilitating a routing (e.g., a physical routing) of one or more objects. The routing may include routing the object(s) to or away from a given processing line or storage bin. For example, in the illustration of FIG. 1, a routing may guide one or more objects to a clean processing line 150a, a dirty processing line 150b, or a Pyromellitic dianhydride (PMDA) additive line 150c.

Routing the object(s) may include (for example) moving one or more robotic arms, each of which may move in a straight and/or angular direction (e.g., along a particular trajectory of one or more predefined particular trajectories). The robotic arm(s) may move in a manner that pushes the object(s) in a target direction, that picks up and moves the object(s) to a target location, that induces a force (e.g., a wind or magnetic force that attracts the object(s) towards a target location, etc.). FIG. 1 illustrates an instance where each of a set of objects represented in the image data 115 is routed to one of three processing lines 150a-150c based on the corresponding action instruction 145. In the exemplary instance, the three processing lines corresponds to a first processing line (e.g. the clean processing line 150a) that is to include objects (or object groups) with at least a threshold predicted amount (e.g., an absolute or percentage threshold amount) of one or more target materials that feeds to a belt to facilitate pyrolysis process 155, a second processing line (e.g. the dirty processing line 150b) that feeds to a an additive-inclusive storage bin 160 for objects or object groups that are predicted to have at least a threshold amount of a given additive (e.g., at least 0.5% of PMDA), and a processing line that feeds to a dirty storage bin 165 for other objects. It will be appreciated that, while FIG. 1 depicts an instance where the first processing line (e.g. the clean processing line 150a) feeds to a belt to facilitate pyrolysis process 155, the belt may feed to an additional or alternative type of downstream processing. Such downstream processing may be configured to (for example) extract one or more select materials (e.g., by performing a dissolution, depolymerization and/or conversion technique).

It will be appreciated that outputs from the pyrolysis (or other downstream) process that are performed on objects fed by the belt to the pyrolysis process 155 depend on the materials that are in those objects. For example, for a pyrolysis process, objects are subjected to high temperatures in the absence of oxygen, such that solid objects are transformed into a pyrolysis liquid. Various catalysts may also be used to enhance the pyrolysis process. The pyrolysis liquid may then be used to produce new objects.

The utility of the pyrolysis liquid depends on the degree to which the pyrolysis liquid can be reliably generated in a manner such that the pyrolysis liquid has consistent and predictable properties. For example, it is advantageous to be able to predict the distribution of various types of molecules in the liquid, the pour point, the density, etc. However, reliably producing a pyrolysis liquid with particular properties can be difficult given the large variety of objects (e.g., plastic products) that may be received in initial feedstocks. For example, each plastic object may include different percent compositions of one or more of: polyvinyl chloride (PVC), polyethylene terephthalate (PET), Low Density Polyethylene (LDPE), High Density Polyethylene (HDPE), polypropylene (PP) and polystyrene (PS). Suppose that a first batch of objects includes more LDPE and less HDPE as compared to a second batch. A result is that new objects produced from a pyrolysis oil from the first batch will have lower strength and less heat resistant as compared to new objects produced from a pyrolysis oil from the second batch. Further, the temperature that can be used to transform the first batch of objects into a liquid would be lower than a temperature that can be used to transform the second batch of objects into a liquid.

Additionally, various objects may include non-plastic materials, such as food residue or product labels that include paper. These non-plastic materials can result in (for example) more char being produced by a pyrolysis process, which can be undesirable.

Therefore, in some embodiments, hyperspectral data is used to generate one or more action instructions to facilitate reliable production of consistent pyrolysis liquids. The action instructions may be generated by (for example) using pyrolysis model to predict one or more characteristics of a pyrolysis process if it is receiving, as input, a particular set of objects (e.g., being routed towards the pyrolysis processing line). For example, composition prediction data 140 corresponding to the particular set of objects can be aggregated and fed to the pyrolysis model to predict characteristics of outputs of the pyrolysis process. Such predictions may include amounts of oil, gas, and/or char that would be produced or that characterizes particular properties of the oil. Characteristics of the oil can include (for example) its: boiling point, liquid yield, viscosity, density, halogen count, chlorine count, contribution of one or more types of chemicals (e.g., N-paraffins, N-olefins, iso-paraffins, iso-olefins, cyclo-olefins, and/or aromatics). In some instances, feature engineering is performed to identify particular features to feed to the pyrolysis model. The pyrolysis model may include (for example) a neural network, regression model, support vector machine, component analysis (e.g., principal component analysis), decision-tree model, etc. The pyrolysis model may include a model pretrained for another use case that was then fine-tuned to predict the characteristic(s) of outputs of the pyrolysis process.

In some instances, the hyperspectral data includes data generated based on the image data 115 collected by the camera system 110 For example, by using the segmentation data 135 and action instructions 145, the computing system 130 can infer which portions of collected hyperspectral data correspond to objects routed—within an iteration or time interval—to the belt to the pyrolysis process 155. As another example, by using the segmentation data 135, composition prediction data 140, and action instructions 145, the computing system 130 can infer a per-object or cumulative composition data for objects on the belt to the pyrolysis process 155 at a given point in time. The computing system 130 can then generate one or more action instructions based on per-object or cumulative composition data.

In some instances, another camera system 170 is positioned to image downstream of the camera system 110. For example, the other camera system 170 may be positioned to collect images of a portion of the clean processing line 150a. The other camera system 170 may include one or more characteristics as disclosed herein with respect to the camera system 110. Image data collected by the other camera system 170 may have one or more characteristics as disclosed herein with respect to the image data 115. In some instances, the other image data collected by the other camera system 170 is availed to the computing system 130 or another computing system. Optionally, the computing system 130 or the other computing system may use the other image data to perform a segmentation technique (e.g., as disclosed herein) and/or may generate composition prediction data for one or more objects (e.g., as disclosed herein). The computing system 130 or the other computing system may generate one or more action instructions based on (for example) the image data, the segmentation data, and/or the composition prediction data.

Action instructions 145 (e.g., whether generated based on the image data 115 collected by the camera system 110 or based on other image data collected by the other camera system) may include (for example) a sorting instruction, a routing instruction, an instruction for a parameter of a downstream processing, an instruction for criteria to use for a subsequent sorting, etc.

For example, FIG. 1 illustrates an instance where objects from the clean processing line 150a may be either routed to the belt to the pyrolysis process 155 or to the dirty storage bin 165. An action instruction may indicate how each of one or more objects are to be routed (e.g., whether to the belt to the pyrolysis process 155 or the dirty storage bin 165) or how a set of objects (e.g., all objects depicted in image, all objects fully depicted in an image, all objects with at least a threshold number of pixels in an image, etc.) are to be routed. A robotic arm may then be used to selectively lift, slide, push or otherwise move each object (or each object corresponding to a particular route) or to move each set of objects appropriately. For example, in one instance the clean processing line 150a includes a belt that moves towards the belt to the pyrolysis process 155. Thus, any object that is to be routed to the belt to the pyrolysis process 155 may be carried there by default, whereas a robotic arm may move any object that is not to be routed to the belt to the pyrolysis process 155 (e.g., and is instead to be routed to the dirty storage bin 165) to a different route or location.

Therefore, individual objects and/or individual sets of objects may be dynamically routed towards or away from a downstream processing line based on (for example) hyperspectral data, a machine-learning algorithm that predicts composition attributes of objects, and/or estimated properties of objects in a current or upcoming batch for the downstream processing.

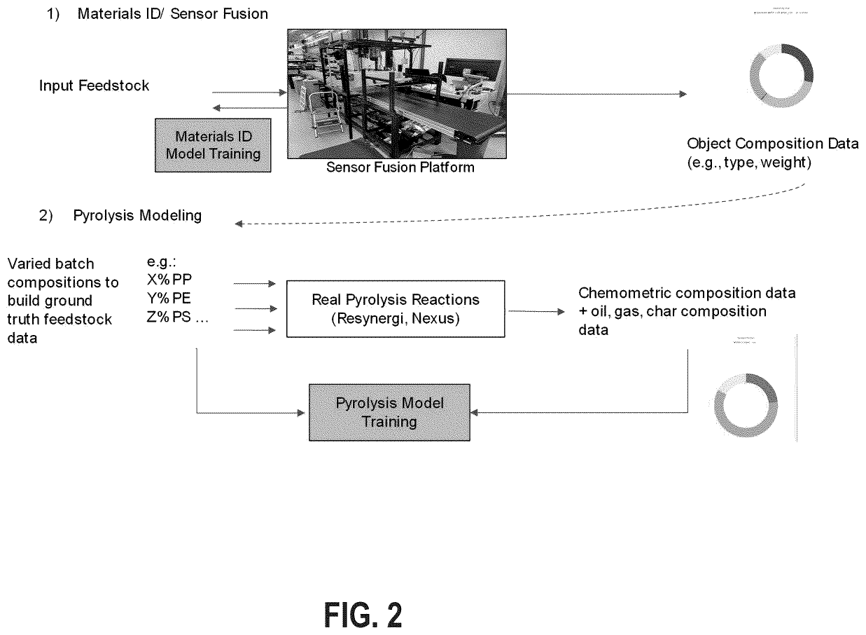

FIG. 2 illustrates a process stream where a machine-learning model is trained to process image data to predict characteristics of feedstocks. It will be appreciated that one or more actions represented in the process stream of FIG. 2 may correspond to one or more actions related to FIG. 1, though the scope and/or specifics of such actions may potentially vary. In the instance, an input feedstock is received, and one or more sensors collect sensor data (e.g., image data) for at least part of the input feedstock (e.g., for an individual object or for a set of objects). For example, one or more cameras may collect one or more hyperspectral images (e.g., corresponding to one or more wavelength bands) of the object(s). The input feedstock may include outputs from an initial sorting (e.g., a manual and/or automated sorting). The input feedstock may include objects predicted to have one or more particular types of materials. To illustrate, the input feedstock may include plastics of or including any of #3-#7 (including polyvinyl chloride, low-density polyethylene, polypropylene, polystyrene, BPS, polycarbonate, or LEXAN).

A computing system (not shown) can use the sensor data to generate composition prediction data, which may include (for example) an identification of one or more materials in the object, a material contribution of particular material or particular type of material in the object(s), etc. The composition prediction data may include object composition data that includes data predicting (for example) which materials are in the object(s), whether a given material is in the object(s), a portion of the object's/objects' weight or mass attributable to a particular material, which material types are in the object(s), whether a given material type is in the object(s), a portion of a weight of mass of the object(s) attributable to a particular material type, etc. The object composition data may further and/or alternatively predict a weight or mass of the object(s); a weight or mass of one or more particular materials; and/or a weight or mass of one or more particular types of materials. Part or all of the object composition data may be generated by processing one or more images (e.g., one or more hyperspectral images) of the object(s) using a machine-learning model.

Using object composition data corresponding to a batch of objects, a predicted distribution of materials in the batch can be generated. For example, FIG. 2 illustrates predicting percentages of the predicted masses of the multiple objects that are predicted to be PP, PE or PS. The batch can then be processed by a real pyrolysis reaction, and physical outputs from the reaction can be analyzed. For example, the analysis may include chemometric composition data that characterizes amounts of oil, gas and/or char that were produced or that characterizes particular properties of the oil. Characteristics of the oil can include (for example) its: boiling point, liquid yield, viscosity, density, halogen count, chlorine count, contribution of one or more types of chemicals (e.g., N-paraffins, N-olefins, iso-paraffins, iso-olefins, cyclo-olefins, and/or aromatics).

The chemometric composition data can then be used (e.g., along with the object composition data and/or the predicted distribution of materials in the batch) to train or fine-tune a pyrolysis model. Once trained, the pyrolysis model can then later be used to transform an input data set that includes predicted amounts of one or more materials in a batch to predictions as to the amount of or characteristics of various outputs of a pyrolysis process.

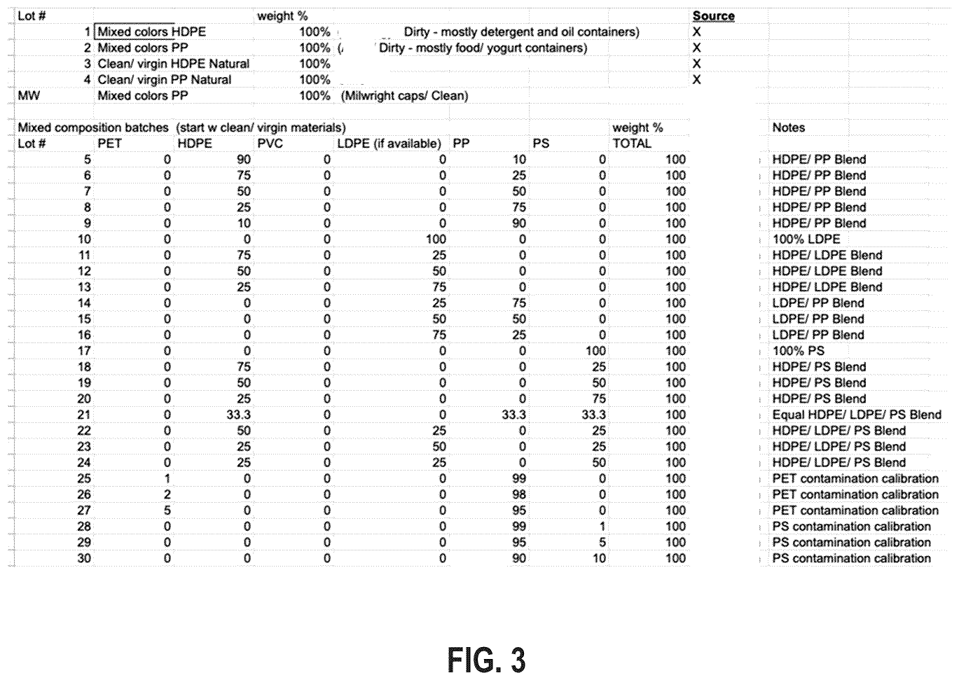

In some instances, batches used to generate training data are strategically designed. For example, various batches may be designed to include particular distributions of materials that may facilitate generating accurate predictions across an entire multi-dimensional space of interest. For example, FIG. 3 identifies exemplary specifications for 30 batches for training, where the batches include different relative amounts of HDPE, LDPE, PP, PS and contaminants. Notably, these batches do not include PET or PVC. This may be because a corresponding use case is one where a sorting occurs to divert objects from the processing line when the objects include PET or PVC.

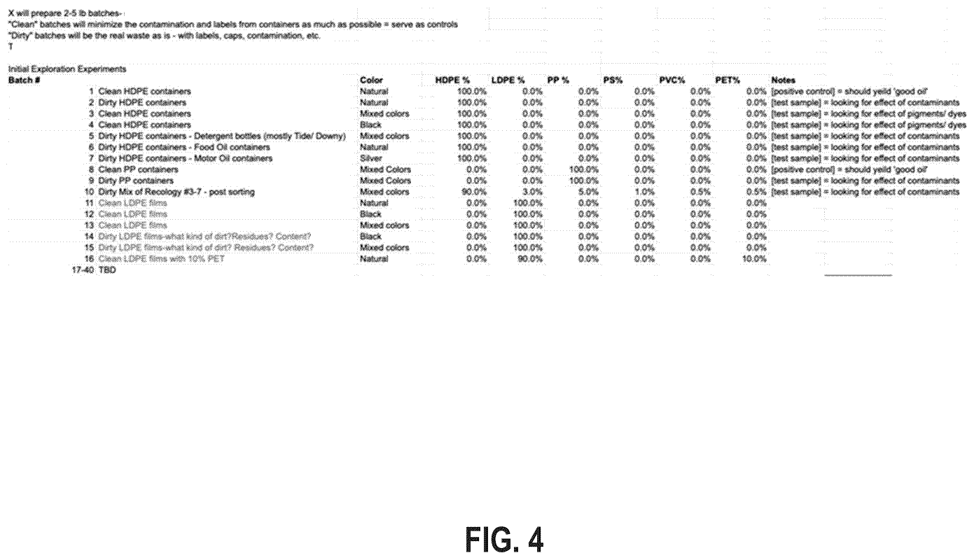

As another example, FIG. 4 identifies exemplary specifications for 16 batches for training. In this instance, the batches include different types of colors and different relative amounts of HDPE, LDPE, PP, PVC and PET. Notably, these batches do not include PVC. Some of the batches in this example are “clean”, while others are “dirty”. The dirty batches include different types of contaminants (e.g., food oil, motor oil, detergent, etc.). Use of the dirty batches may facilitate the model learning about a variety of objects that may be represented at a single point in a multi-dimensional space defined based on relative contributions of various types of materials (e.g., various types of plastics). Accordingly, the model may tailor predictions to account for this variability. Additionally or alternatively, it may be determined that the outputs of processing lines are substantially affected by one or more types of contaminants, in which case a sorting technique may be adjusted to sort based on the presence of or amount of contaminants of the one or more types.

### Example

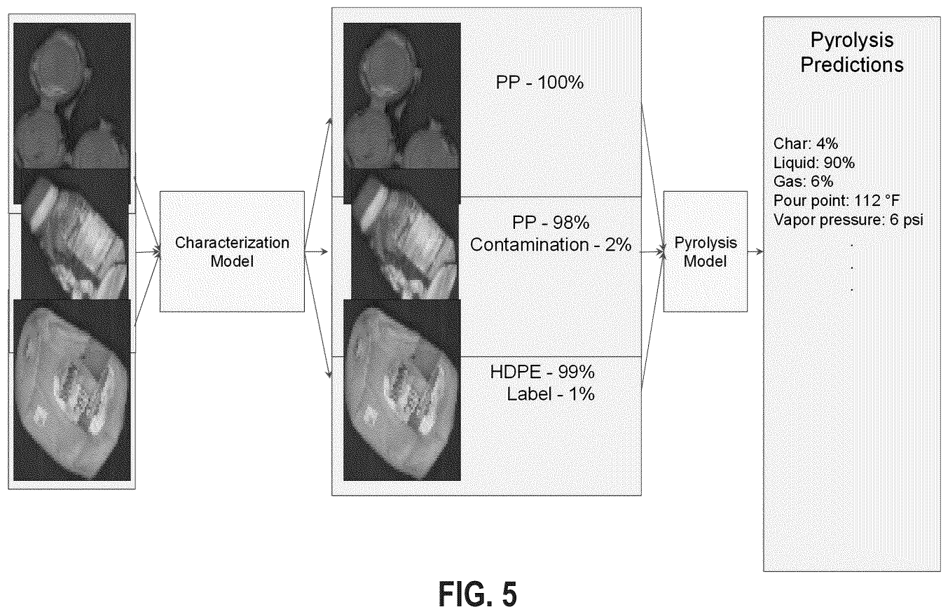

FIG. 5 illustrates an example where three hyperspectral images of objects were used to predict pyrolysis outputs of a batch that includes the images. The three images on the left correspond to image data corresponding to different one or more objects. While a single image is shown for each of the three instances, the images are merely representative, and other image data corresponded to different wavelength bands (thereby corresponding to a hyperspectral cube).

A characterization model included a machine-learning model that detected and excluded background pixels and used the remaining hyperspectral data to predict object composition data for the depicted objects. For example, the characterization model predicted that the object(s) depicted in the top image are completely composed of polypropylene.

The predicted object composition data was then fed to a pyrolysis model, which predicted pyrolysis predictions corresponding to characteristics of predicted outputs of a pyrolysis process if all of the depicted objects were fed to the pyrolysis process in a given batch. The characteristics of the predicted outputs included predictions as to how much of a reaction output would be a pyrolysis liquid, versus char, versus gas. In the illustrated case, it was estimated that the outputs would be 90% pyrolysis liquid, 4% char, and 6% gas.

In this example, the characteristics of the predicted output further included a pour point and vapor pressure of the pyrolysis liquid.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

A number of implementations have been described. Nevertheless, it will be understood that various modifications may be made. For example, various forms of the flows shown above may be used, with steps re-ordered, added, or removed. Also, although several applications of providing incentives for media sharing and methods have been described, it should be recognized that numerous other applications are contemplated. Accordingly, other implementations are within the scope of the following claims.

1. A method comprising:
generating an intermediate data set based on an image of a set of objects, wherein each of the set of objects includes a plastic;
generating a predicted chemometric property of a physical output pyrolysis oil produced by performing a pyrolysis processing of the set of objects using a pyrolysis reactor, wherein the predicted chemometric property is generated by inputting the intermediate data set to a machine-learning model; and
generating a result associated with the set of objects, wherein the result is based on or includes the predicted chemometric property.

2. The method of claim 1, wherein generating the intermediate data set includes generating a hypercube based on a set of line scans of the line scans of the set of objects, wherein a first dimension of the hypercube corresponds to a first spatial dimension in a real-world space, a second dimension of the hypercube corresponds to a second spatial dimension in the real-world space, a third dimension of the hypercube corresponds to a frequency dimension, and values in the hypercube represent at least one of an intensity, a power, a reflectance, a transmittance, an absorbance, and a trans-reflectance.

3. The method of claim 1, wherein generating the intermediate data set includes generating, for each material of a set of materials, a predicted relative or absolute amount of the material in the set of objects.

4. The method of claim 1, wherein generating the intermediate data set includes generating, for each material of a set of materials, a portion of a weight or mass of the set of objects that is predicted to be attributed to the material.

5. The method of claim 1, wherein the predicted chemometric property of the pyrolysis oil is an American Petroleum Institute (API) gravity, density, or relative density of the pyrolysis oil, and wherein an overall quality metric or classifier is derived by an aggregate of an individual chemometric property or other predictive functions.

6. The method of claim 1, wherein the predicted chemometric property of the pyrolysis oil is a vapor pressure of a crude oil produced using the pyrolysis oil.

7. The method of claim 1, wherein the predicted chemometric property of the pyrolysis oil is a pour point of the pyrolysis oil.

8. The method of claim 1, wherein the predicted chemometric property of the pyrolysis oil is or is based on an amount of one or more halogens in the pyrolysis oil.

9. The method of claim 1, wherein the predicted chemometric property of the pyrolysis oil is or is based on an amount of inorganic contaminants and organic contaminants in the pyrolysis oil, wherein the inorganic contaminants comprise at least one of sulfur, chlorine, and phosphorus and the organic contaminants comprise at least one of sulfur, Polyfluorinated Substances (PFAS), caprolactams, organic acids, perflourinated and flourinated compounds, halogenated organic compounds, and oxygen measured by neutron activation.

10. The method of claim 1, further comprising controlling whether the set of objects are routed towards a pyrolysis-process pipeline based on the result.

11. The method of claim 1, wherein the result includes a selection or identification of one or more other objects to combine with the set of objects before the pyrolysis processing is performed on the set of objects.

12. The method of claim 1, wherein the image of the set of objects is generated based on a set of line scans obtained at different wavelengths.

13. The method of claim 12, wherein the wavelengths are selected from a group of wavelength ranges consisting of 1000-1700 nm, 2200-5000 nm, and 400-1000 nm.

14. The method of claim 1, wherein the image of the set of objects is generated by performing one of a line scan, an area scan, and a point mapping.

15. The method of claim 1, wherein generating the intermediate data set includes performing, for each material of a set of materials, hydrocarbon analysis on the set of objects, and wherein the hydrocarbon analysis provides a profile of at least one paraffins, iso-paraffins, and aromatics present in the set of objects.

16. The method of claim 1, wherein generating the intermediate data set includes performing, for each material of a set of materials, simulated distillation of the set of objects, and wherein the simulated distillation provides volumetric distillation profiles of the set of objects.

17. A system comprising:
one or more computers; and
one or more computer-readable media storing instructions that are operable, when executed by the one or more computers, to cause the system to perform a set of actions including:
generating an intermediate data set based on an image of a set of objects, wherein each of the set of objects includes a plastic;
generating a predicted chemometric property of a physical output pyrolysis oil produced by performing a pyrolysis processing of the set of objects using a pyrolysis reactor, wherein the predicted chemometric property is generated by inputting the intermediate data set to a machine-learning model; and
generating a result associated with the set of objects, wherein the result is based on or includes the predicted chemometric property.

18. The system of claim 17, wherein generating the intermediate data set includes generating a hypercube based on a set of line scans of the line scans of the set of objects, wherein a first dimension of the hypercube corresponds to a first spatial dimension in a real-world space, a second dimension of the hypercube corresponds to a second spatial dimension in the real-world space, a third dimension of the hypercube corresponds to a frequency dimension, and values in the hypercube represent at least one of an intensity, a power, a reflectance, a transmittance, an absorbance, and a trans-reflectance.

19. The system of claim 17, wherein generating the intermediate data set includes generating, for each material of a set of materials, a predicted relative or absolute amount of the material in the set of objects.

20. The system of claim 17, wherein generating the intermediate data set includes generating, for each material of a set of materials, a portion of a weight or mass of the set of objects that is predicted to be attributed to the material.

21. The system of claim 17, wherein the predicted chemometric property of the pyrolysis oil is an American Petroleum Institute (API) gravity, density, or relative density of the pyrolysis oil, and wherein an overall quality metric or classifier is derived by an aggregate of an individual chemometric property or other predictive functions.

22. The system of claim 17, wherein the predicted chemometric property of the pyrolysis oil is a vapor pressure of a crude oil produced using the pyrolysis oil.

23. The system of claim 17, wherein the predicted chemometric property of the pyrolysis oil is a pour point of the pyrolysis oil.

24. The system of claim 17, wherein the predicted chemometric property of the pyrolysis oil is or is based on an amount of one or more halogens in the pyrolysis oil.

25. One or more non-transitory computer-readable media storing instructions that are operable, when executed by one or more computers, to cause a system to perform a set of actions including.
generating an intermediate data set based on an image of a set of objects, wherein each of the set of objects includes a plastic;
generating a predicted chemometric property of a physical output pyrolysis oil produced by performing a pyrolysis processing of the set of objects using a pyrolysis reactor, wherein the predicted chemometric property is generated by inputting the intermediate data set to a machine-learning model; and
generating a result associated with the set of objects, wherein the result is based on or includes the predicted chemometric property.

26. The method of claim 12, wherein the wavelengths are above 2200 nm.
